(19) United States
(12) Patent Application Publication
(10) Pub. No.: US 2024/0260995 A1
(43) Pub. Date: Aug. 8, 2024

(54) BONE POSITIONING AND CUTTING SYSTEM AND METHOD

(71) Applicant: Treace Medical Concepts, Inc., Ponte Vedra, FL (US)

(72) Inventors: Paul Dayton, Ankeny, IA (US); F. Barry Bays, Collierville, TN (US)

(21) Appl. No.: 18/615,875

(22) Filed: Mar. 25, 2024

Related U.S. Application Data
(63) Continuation of application No. 17/503,730, filed on Oct. 18, 2021, now Pat. No. 11,937,849.
Application No. 17/503,730 is a continuation of application No. 17/201,008, filed on Mar. 15, 2021, now Pat. No. 11,147,590.
Application No. 17/201,008 is a continuation of application No. 16/730,424, filed on Dec. 30, 2019, now Pat. No. 10,945,764.
Application No. 16/730,424 is a continuation of application No. 15/894,702, filed on Feb. 12, 2018, now Pat. No. 10,555,757.
Application No. 15/894,702 is a division of application No. 14/799,981, filed on Jul. 15, 2015, now abandoned.
(60) Provisional application No. 62/024,546, filed on Jul. 15, 2014.

(51) Int. Cl. A61B 17/64; A61B 17/15; A61B 17/66

(52) U.S. Cl. CPC A61B 17/6416; A61B 17/151; A61B 17/66

(57) ABSTRACT

A bone positioning device can include a fixation pin for attachment to a first bone and a fixation pin for attachment to a second bone. A first block having an aperture can be included for slidably receiving a fixation pin, and a second block having an aperture can be included for slidably receiving a fixation pin. A multi-axis joint can connect the first block and the second block, where the multi-axis joint allows the first second blocks to move with respect to each other about more than one axis.

### RELATED APPLICATIONS

This application is a continuation application of U.S. patent application Ser. No. 17/503,730, filed Oct. 18, 2021, which is a continuation application of U.S. patent application Ser. No. 17/201,008, filed Mar. 15, 2021, issued as U.S. Pat. No. 11,147,590, on Oct. 19, 2021, which is a continuation application of U.S. patent application Ser. No. 16/730,424, filed Dec. 30, 2019, issued as U.S. Pat. No. 10,945,764, on Mar. 16, 2021, which is a continuation application of U.S. patent application Ser. No. 15/894,702, filed Feb. 12, 2018, issued as U.S. Pat. No. 10,555,757, on Feb. 11, 2020, which is a divisional application of U.S. patent application Ser. No. 14/799,981, filed Jul. 15, 2015, which claims priority to U.S. Provisional Application No. 62/024,546, filed Jul. 15, 2014. The entire contents of each of these applications are hereby incorporated by reference.

### TECHNICAL FIELD

This disclosure generally relates to devices and methods for positioning and cutting bones.

### BACKGROUND

In various surgical procedures, it can be necessary to cut one or more bones. Success of such surgical procedures may often times be a function of the accuracy of the cut(s) being made to the one or more bones. Accomplishing accurate cuts can be especially complicated where surgical procedures involve cutting one or more bones that are relatively small as compared to bones in other locations of a surgical patient's anatomy. Exemplary surgical procedures involving cuts to one or more relatively small bones can include surgical procedures involving a foot or hand. To help facilitate accurate cuts to one or more bones, it may be useful to position the one or more bones to be cut in a manner that is conducive to a particular cut.

### SUMMARY

One embodiment includes a bone positioning device. The embodiment of the bone positioning device can include at least one fixation pin for attachment to a first bone and at least one fixation pin for attachment to a second bone. A first block having at least one aperture can be included for slidably receiving a fixation pin(s), and a second block having at least one aperture can be included for slidably receiving a fixation pin(s). A multi-axis joint can connect the first block and the second block, where the multi-axis joint allows the first and second blocks to move with respect to each other about more than one axis.

Another embodiment includes a method for fixing an orientation of a first bone with respect to a second bone. The embodiment of the method can include attaching at least one fixation pin to a first bone and attaching at least one fixation pin to a second bone. At least one fixation pin can be inserted within a respective aperture of a first block, and at least one fixation pin can be inserted within a respective aperture of a second block. The first block can be positioned along and about the fixation pin(s) and a set screw(s) can be actuated to fix a position of the first block along and about the fixation pin(s), and similarly the second block can be positioned along and about the fixation pin(s) and a set screw(s) can be actuated to fix a position of the second block along and about the fixation pin(s). The position of the first block can be adjusted with respect to the second block about at least a first axis and a second axis. A set screw can be actuated to fix a position about the first axis, and a set screw can be actuated to fix a position about the second axis.

A further embodiment includes a bone cutting guide. The embodiment of the bone cutting guide can include a plate defining a plane, a block having a guiding surface integral with or coupled to the plate, with the guiding surface being parallel to the plane and being spaced laterally therefrom. A handle can also be included extending from the plate.

### BRIEF DESCRIPTION OF THE DRAWINGS

The following drawings are illustrative of particular embodiments of the present invention and therefore do not limit the scope of the invention. The drawings are not necessarily to scale (unless so stated) and are intended for use in conjunction with the explanations in the following detailed description. Embodiments of the invention will hereinafter be described in conjunction with the appended drawings, wherein like numerals denote like elements.

FIG. 1 is a perspective view of a bone positioning device according to an embodiment of the invention;

FIG. 2 is a top plan view of the bone positioning device of FIG. 1;

FIG. 3 is a side plan view of the bone positioning device of FIG. 1;

FIG. 4 is a perspective cross-sectional view of the bone positioning device of FIG. 1;

FIG. 5 is a perspective view of a bone positioning device attached to bones in a skewed position according to an embodiment of the invention;

FIG. 6 is a top view of the bone positioning device of FIG. 5;

FIG. 7 is a side view of the bone positioning device of FIG. 5;

FIG. 8 is a side view of the bone positioning device of FIG. 5;

FIG. 9 is a perspective view of a bone cutting guide according to an embodiment of the invention;

FIG. 10 is a perspective view of a bone cutting guide according to an embodiment of the invention in contact with a saw blade; and

FIG. 11 is a side plan view of the bone cutting guide and saw blade of FIG. 10.

### DETAILED DESCRIPTION

The following detailed description is exemplary in nature and is not intended to limit the scope, applicability, or configuration of the invention in any way. Rather, the following description provides some practical illustrations for implementing exemplary embodiments of the present invention. Examples of constructions, materials, dimensions, and manufacturing processes are provided for selected elements, and all other elements employ that which is known to those of ordinary skill in the field of the invention. Those skilled in the art will recognize that many of the noted examples have a variety of suitable alternatives.

Embodiments of the invention include a bone positioning device. Embodiments of the bone positioning device can be useful for temporarily fixing bones in a desired position during a surgical procedure, such as a bone alignment, osteotomy, and/or fusion procedure. Such a procedure may be performed, for example, on bones (e.g., adjacent bones separated by a joint or different portions of a single bone) in the foot or hand. In one example, the procedure can be performed to correct an alignment between a metatarsal (e.g. a first metatarsal) and a cuneiform (e.g., a first cuneiform), such as a bunion correction. An example of such a procedure is a Lapidus procedure. In another example, the procedure can be performed by modifying an alignment of a metatarsal (e.g. a first metatarsal). An example of such a procedure is a Basilar metatarsal osteotomy procedure.

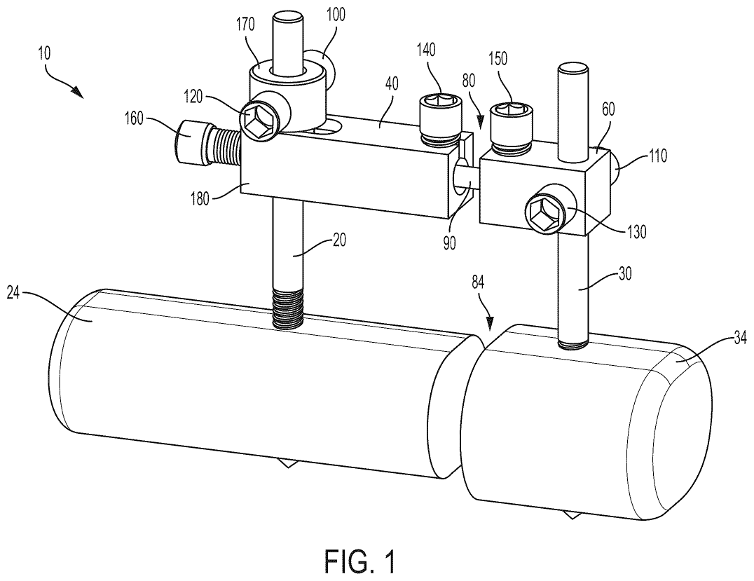

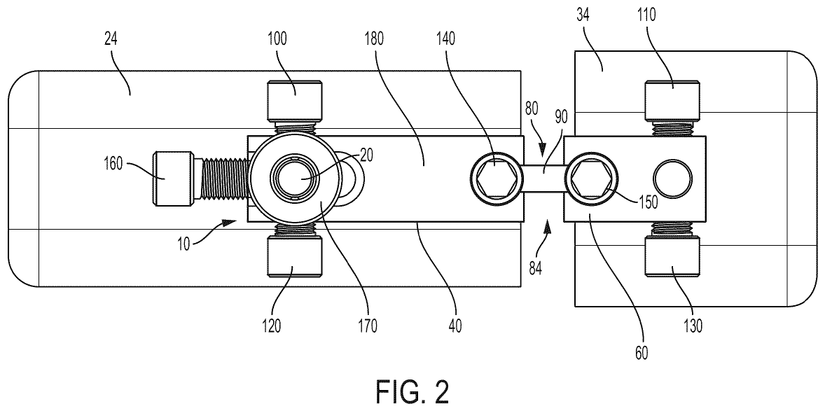

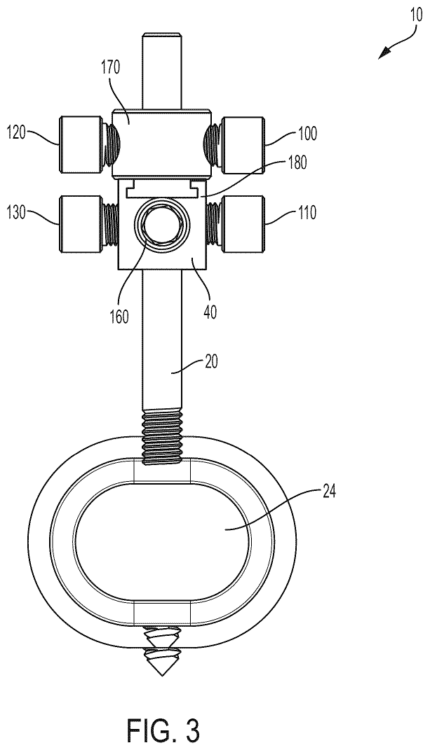

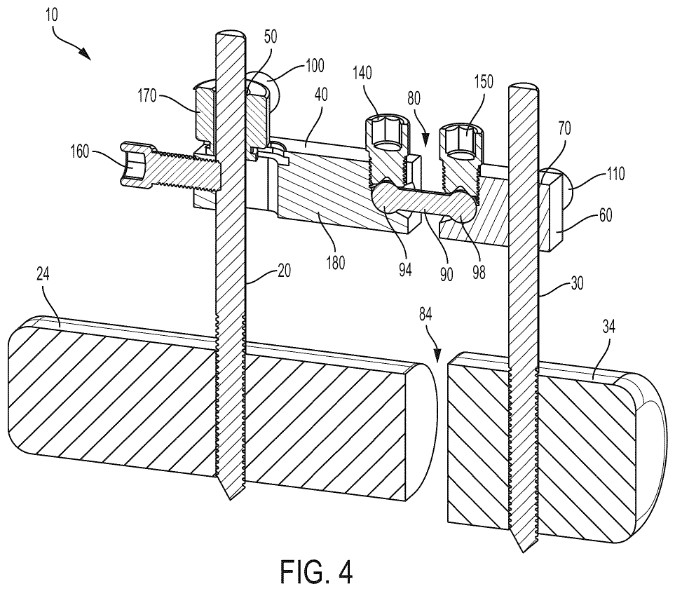

As shown in FIGS. 1-4, the bone positioning device 10 can include at least one fixation pin, such as a first fixation pin 20, for attachment to a first bone 24. At least one fixation pin, such as a second fixation pin 30, can be provided for attachment to a second bone 34, such as an adjacent bone separated by a joint or different portions of a single bone. As shown best in FIG. 4, a first block 40 having a first aperture 50 can slidably receive the first fixation pin 20, and a second block 60 having a second aperture 70 can slidably receive the second fixation pin 30. The first and second apertures 50, 70 can allow the first and second blocks 40, 60 to slide along a longitudinal axis of the first and second fixation pins 20, 30, respectively. The first and second apertures 50, 70 can also allow the first and second blocks 40, 60 to rotate about the longitudinal axis of the first and second fixation pins 20, 30, respectively. In some embodiments, each of the first and second fixation pins 20, 30 are generally cylindrical and have a distal portion and a proximal portion, with the distal portion threaded for retention within the respective first or second bone, while the proximal portion is unthreaded for both sliding within the respective first or second aperture and free rotational movement within the respective first or second aperture. In some embodiments, the proximal portion has a uniform diameter, such that it does not contain a flared or “head” portion. In such embodiments, the first and second blocks can be positioned on the first and second fixation pins before or after the pins are engaged with bone.

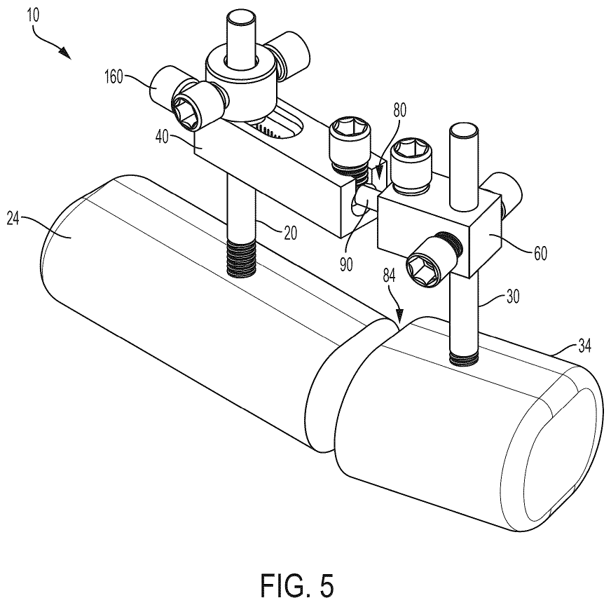

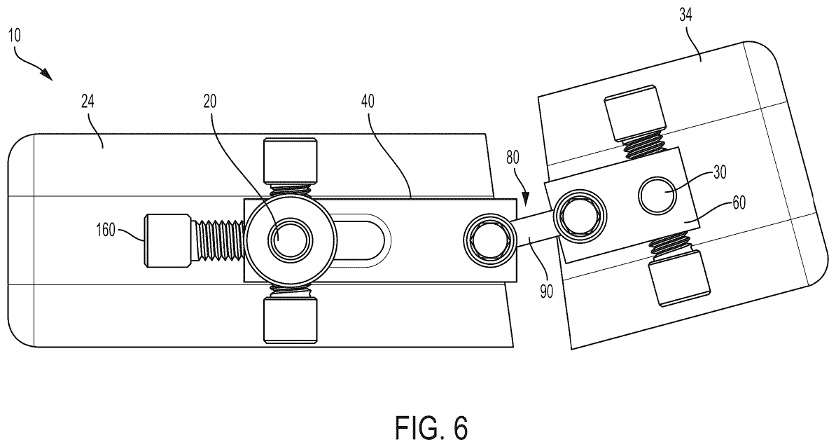

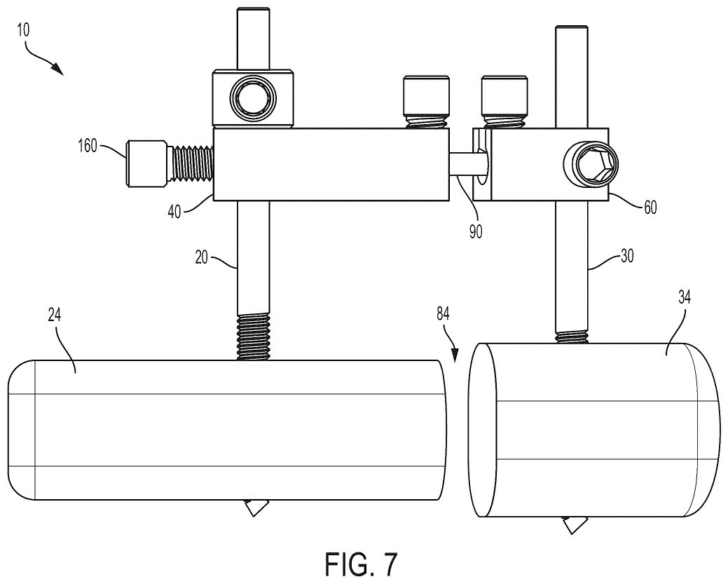

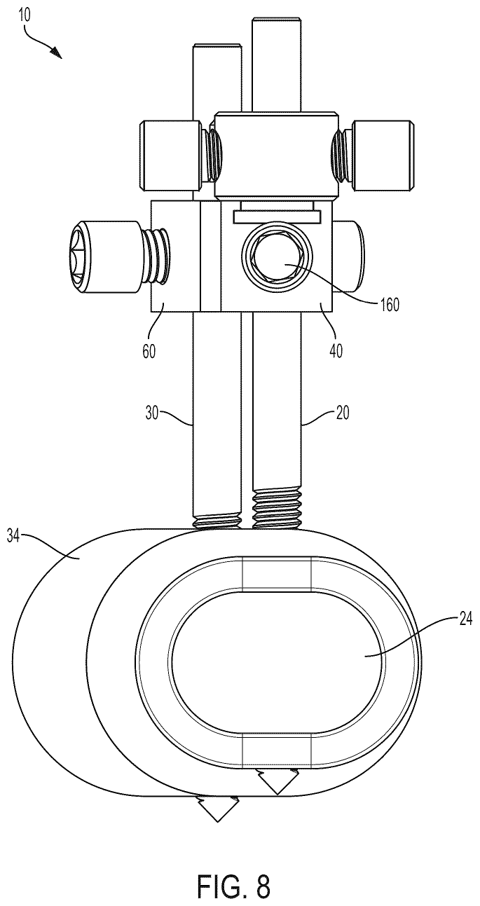

Again as shown best in FIG. 4, a multi-axis joint 80 can be provided to connect the first block 40 and the second block 60 and located adjacent to a joint 84 between the first and second bones. In some embodiments, the multi-axis joint 80 allows the first block 40 and the second block 60 to move with respect to each other about more than one axis. In certain embodiments, the multi-axis joint 80 allows the first block 40 and the second block 60 to move with respect to each other about the three cardinal planes (i.e., X, Y, and Z axes). In the embodiment shown, the multi-axis joint 80 allows for angulation in all directions and rotation between the first and second blocks. FIGS. 5-8 depict an exemplary embodiment of the bone positioning device 10 attached to first and second bones 24, 34, where the first and second bones are skewed relative to each other. In this particular embodiment, a longitudinal axis of second bone 34 is skewed about 15 degrees relative to a longitudinal axis of first bone 24.

The multi-axis joint can include any suitable structure for allowing desired adjustments about more than one axis, such as desired adjustments about three axes. In some embodiments, with reference to FIG. 4, the multi-axis joint 80 includes a link 90 having a first end 94 rotatably connected to the first block 40 and a second end 98 rotatably connected to the second block 60. Such a multi-axis joint allows for the movement about the various axes discussed above at both the first end and the second end. In the embodiment shown, the first end 94 includes a first ball received within a first socket of the first block 40, and the second end 98 includes a second ball received within a second socket of the second block 60.

Some embodiments of the device allow the relative positions of the first and second bones to be fixed after a desired orientation has been achieved. For example, a first set screw 100 can extend through the first block 40 into the first aperture 50 and be positioned against the first fixation pin 20, for fixation of the first block on a longitudinal axis of the first fixation pin and/or about the longitudinal axis of the first fixation pin. Further, a second set screw 110 can extend through the second block 60 into the second aperture 70 and be positioned against the second fixation pin 30, for fixation of the second block on a longitudinal axis of the second fixation pin and/or about the longitudinal axis of the second fixation pin. In certain embodiments, the first and second set screws are positioned perpendicular to the first and second fixation pins. As shown in FIGS. 1-3, additional set screws 120, 130 extending through the first and second blocks can be positioned opposite of the first and second set screws, respectively. Such oppositely positioned set screws may facilitate use of the bone positioning device on a left foot or a right foot depending on a particular surgical procedure.

Set screws can also be provided to fix positions across the multi-axis joint. In the embodiment shown in FIG. 4, a first end set screw 140 extends through the first block 40 and is positioned against the first end 94 of the link 90. Further, a second end set screw 150 is shown extending through the second block 60 and positioned against the second end 98 of the link 90.

The set screws can include any structure suitable to fix the relative positions of the components described herein. In some embodiments, the set screws have a threaded connection with the blocks. Further, as shown, they can include a recess with a non-circular surface. Such a recess is useful for engagement with a driving tool, such as a hex-driver.

In some embodiments, the device can be used to apply a compression force between two adjacent bones, or different portions of a single bone, while the bones are held in desired alignment and/or to facilitate a desired alignment between the bones. Such a compression force is useful for certain surgical procedures, such as bone fusions. As shown in FIG. 4, in some embodiments the device 10 includes a compression screw 160 operable to exert a compression force between first and second bones 24, 34 connected to first and second fixation pins 20, 30, respectively. In the embodiment shown, the compression screw 160 is generally perpendicular to the fixation pins and is threadingly received within a block and positioned to act against one of the fixation pins.

One of the blocks can be adapted to allow for relative movement to exert the compression force. In the embodiment shown in FIG. 4, one of the blocks (e.g., the first block 40) has a first portion 170 slidingly connected to second portion 180. An aperture (e.g., the first aperture 50) extends through the first portion and the second portion. In this embodiment, the first aperture has a first cross-sectional area in the first portion and a second cross-sectional area in the second portion, and the first cross-sectional area is smaller than the second cross-sectional area. The set screw 100 can extend through the first portion 170. The compression screw 160 can extend through the second portion 180. Upon actuation, the compression screw 160 will act against the fixation pin 20 and will pull the second portion 180 of the block 40 away from the fixation pin 20. The force will be transmitted through the multi-axis joint 80 through the other block 60 and fixation pin 30, thereby applying a compression force that tends to press together leading surfaces (e.g., interfacing surfaces) of the first and second bones 24, 34.

Some embodiments include a bone cutting guide. Such a guide can be useful for guiding the cutting of bone, such as after a position of the bone has been fixed by the device described above. Bone cutting may be useful, for example, to facilitate contact between leading edges of adjacent bones or different portions of a single bone, such as in a bone alignment and/or fusion procedure.

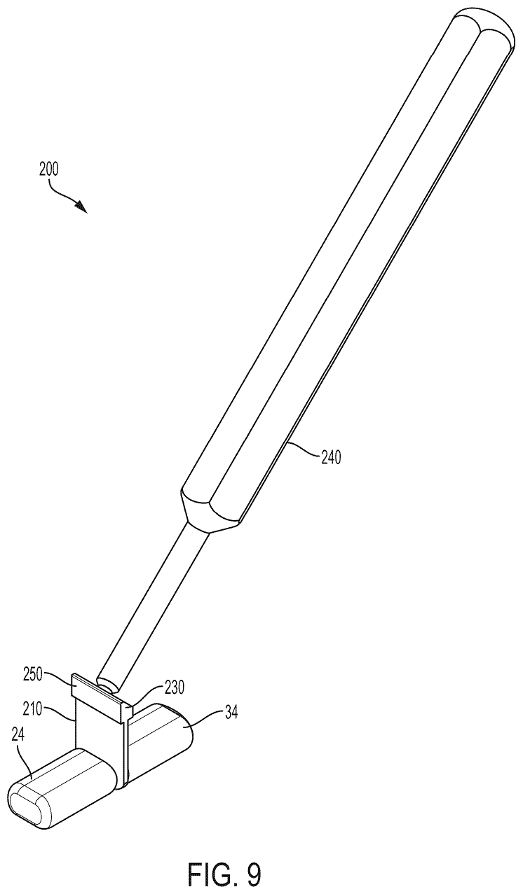

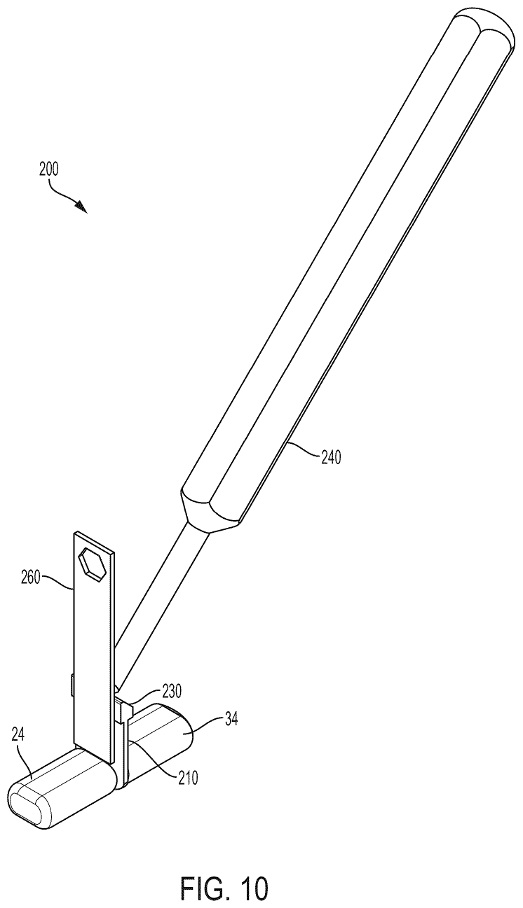

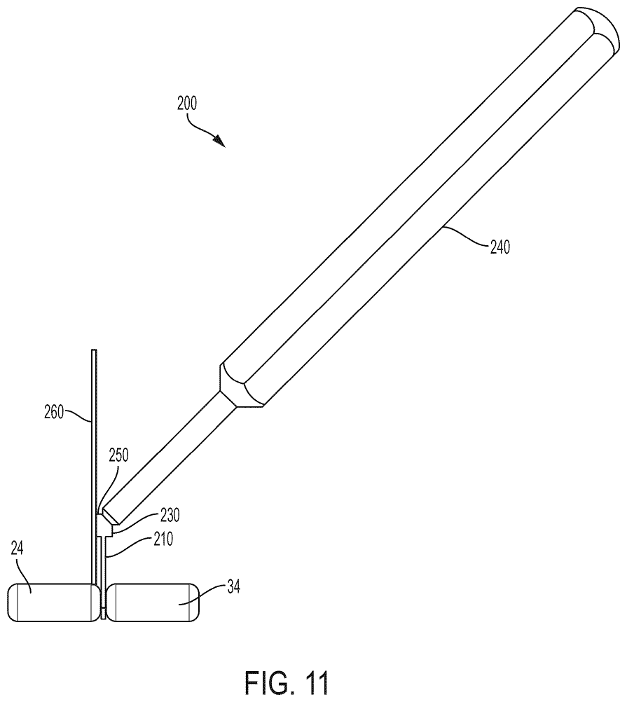

An embodiment of a bone cutting guide is shown in FIGS. 9-11. In the embodiment shown, the guide 200 includes a plate 210 that defines a plane, a block 230, and a handle 240 extending from the block 230. As shown, the handle extends from the block 230 at an angle of between 30 and 60 degrees (e.g., 45 degrees) with respect to the plane. The plate 210 can include a top edge, a bottom edge opposite of the top edge, and first and second opposite side surfaces. In the embodiment shown in FIG. 9, the block 230 can include a surface 250 parallel to the plate 210 and offset from the plate by a distance (e.g., 1-20 millimeters). As shown in FIGS. 10 and 11, a cutting tool 260, such as a saw blade, may be placed in apposition to the surface 250 to guide cutting in a plane parallel to the plate and offset from it by a distance (e.g., a distance the surface 250 if offset from the plate 210).

In use, the bottom edge of the plate 210 can be placed such that it extends into a joint space or resected portion between the first bone 24 and a second bone 34. The surface 250 can provide a cutting tool guide surface operable to guide a cutting tool to cut a leading edge of a bone in a plane parallel to the plate 210.

Embodiments of the invention also include methods of temporarily fixing the orientation of a first bone with respect to a second bone, such as during a surgical procedure, using a bone positioning device. In some embodiments, the method includes a step of attaching a first fixation pin slidably and rotatingly received within a first aperture of a first block to a first bone and attaching a second fixation pin slidably and rotatingly received within a second aperture of a second block to a second bone. The method can also include the steps of positioning the first block along the first fixation pin and actuating a first set screw to fix a position of the first block along the first fixation pin. Likewise, the method can include the steps of positioning the second block along the second fixation pin and actuating a second set screw to fix a position of the second block along the second fixation pin. In some embodiments, the method can include the steps of adjusting the position of the first block with respect to the second block about at least a first axis and a second axis and actuating a third set screw to fix a position about the first axis and actuating a fourth set screw to fix a position about the second axis. In certain embodiments, the method can also include actuating a compression screw to apply a compression force between the first and second bones. It should be noted these steps need not be performed in the order stated, which is merely exemplary. For example, the second fixation pin may be attached to the second bone before the first fixation pin is attached to the first bone, both fixation pins may be attached before either block is adjusted or fixed, etc.

The method may also include steps following the fixing of the position of the bones. Some embodiments of the method also include imaging (e.g., with an X-ray) the first and second bones connected to the first and second blocks to confirm a desirable alignment. Certain embodiments of the method include fusing the first bone and the second bone, such as by attaching a bone connector (e.g., a plate, pin, screw, wire, or staple) to stably connect and fix the first bone and the second bone. Some embodiments also include the step of removing the first fixation pin from the first bone and the second fixation pin from the second bone, such as at a time after the bones have been stabilized and connected with a bone connector.

Some embodiments of the method also include cutting a leading edge of the first or second bone using a cutting guide, such as by positioning a cutting guide proximate the bone (e.g., within a joint between adjacent bones or a resected portion of a single bone) and using the guide to cut a leading edge of the bone in a plane. Such embodiments can also include the step of actuating a compression screw to apply a compression force between the first and second bones after the cutting step.

Thus, embodiments of the invention are disclosed. Although the present invention has been described with reference to certain disclosed embodiments, the disclosed embodiments are presented for purposes of illustration and not limitation and other embodiments of the invention are possible. One skilled in the art will appreciate that various changes, adaptations, and modifications may be made without departing from the spirit of the invention.

What is claimed is:

1. A bone positioning device, comprising:
a first fixation pin for attachment to a first bone;
a second fixation pin for attachment to a second bone;
a first block having a first aperture for slidably receiving the first fixation pin;
a second block having a second aperture for slidably receiving the second fixation pin; and
a multi-axis joint connecting the first block and the second block, the multi-axis joint allowing the first block and the second block to move with respect to each other about more than one axis.

2. The bone positioning device of claim 1, wherein the multi-axis joint further includes a link having a first end rotatably connected to the first block and a second end rotatably connected to the second block.

3. The bone positioning device of claim 2, wherein the first end includes a first ball received within a first socket of the first block, and the second end includes a second ball received within a second socket of the second block.

4. The bone positioning device of claim 2, further including a first end set screw extending through the first block and positioned against the first end.

5. The bone positioning device of claim 2, further including a second end set screw extending through the second block and positioned against the second end.

6. The bone positioning device of claim 1, further including a first set screw extending through the first block into the first aperture and positioned against the first fixation pin, and a second set screw extending through the second block into the second aperture and positioned against the second fixation pin.

7. The bone positioning device of claim 6, wherein the first and second set screws are positioned perpendicular to first and second fixation pins, respectively.

8. The bone positioning device of claim 1, further including a compression screw operable to exert a compression force between the first and second fixation pins.

9. The bone positioning device of claim 8, wherein the compression screw is positioned against one of the first or second fixation pins.

10. The bone positioning device of claim 8, wherein the first or second block has a first portion slidingly connected to second portion, the first aperture extending through the first portion and the second portion, the first aperture having a first cross-sectional area in the first portion and a second cross-sectional area in the second portion, the first cross-sectional area being smaller than the second cross-sectional area, and the compression screw extending through the second portion.

11. The bone positioning device of claim 10, wherein the first or second set screw extends through the first portion.

12. A method of fixing the orientation of a first bone with respect to a second bone, comprising the steps of:
attaching a first fixation pin to a first bone;
attaching a second fixation pin to a second bone;
inserting the first fixation pin within a first aperture of a first block;
inserting the second fixation pin within a second aperture of a second block;
positioning the first block along and about the first fixation pin;
actuating a first set screw to fix a position of the first block along and about the first fixation pin
positioning the second block along and about the second fixation pin;
actuating a second set screw to fix a position of the second block along and about the second fixation pin;
adjusting the position of the first block with respect to the second block about at least a first axis and a second axis;
actuating a third set screw to fix a position about the first axis; and
actuating a fourth set screw to fix a position about the second axis.

13. The method of claim 12, further including the step of actuating a compression screw to apply a compression force between the first and second bones and facilitate a desired alignment between first and second bones.

14. The method of claim 12, further including the step of imaging the first and second bones connected to the first and second blocks.

15. The method of claim 12, further including the step of positioning a cutting guide proximate a leading edge of the first or second bone, and cutting the bone with a cutting tool guided by the cutting guide.

16. The method of claim 15, further including the step of actuating a compression screw to apply a compression force between the first and second bones after the cutting step and facilitate a desired alignment between first and second bones.

17. The method of claim 12, further including the step of removing the first fixation pin from the first bone and the second fixation pin from the second bone after attaching a bone connector to the first and second bones.

18. The method of claim 12, wherein the first bone is a metatarsal and the second bone is a cuneiform.

19. The method of claim 12, wherein the first bone is a first portion of a metatarsal and the second bone is a second portion of the metatarsal.

20. A bone cutting guide, comprising;
a plate defining a plane;
a block having a guiding surface integral with or coupled to the plate, the guiding surface being parallel to the plane and being spaced laterally therefrom; and
a handle extending from the plate.
